(19) United States
(12) Patent Application Publication
(10) Pub. No.: US 2024/0226156 A1
(43) Pub. Date: Jul. 11, 2024

(54) ANTI-FIBROTIC TISSUE RESIDENT MEMORY T CELLS AND USES THEREOF

(71) Applicant: THE JOHNS HOPKINS UNIVERSITY, Baltimore, MD (US)

(72) Inventors: Maureen Renee Horton, Baltimore, MD (US); Jonathan D. Powell, Baltimore, MD (US); Samuel L. Collins, Baltimore, MD (US); Yee Choi Chan Li, Baltimore, MD (US)

(21) Appl. No.: 18/404,727

(22) Filed: Jan. 4, 2024

Related U.S. Application Data
(60) Provisional application No. 63/438,458, filed on Jan. 11, 2023.

(51) Int. Cl. A61K 35/17; A61K 9/00; A61K 39/00; A61P 11/00; C07K 14/705

(52) U.S. Cl. CPC A61K 35/17; A61K 9/0073; A61K 39/4611; A61K 39/464429; A61P 11/00; C07K 14/70546; A61K 2239/38

(57) ABSTRACT

Provided herein are T_{RM} cells, compositions thereof and methods of use thereof. The T_{RM} cells inhibit profibrotic gene expression, suppress profibrotic inflammatory conditions, and inhibit and remediate fibrotic pathogenesis. The T_{RM} cells can localize to specific tissues, enabling tissue and organ-targeted treatment. The methods of use include methods of treating and/or preventing fibrosis, including pulmonary fibrosis.

### CROSS REFERENCE TO RELATED APPLICATIONS

The present application claims the benefit of priority under U.S.C. § 119(e) to U.S. Provisional Application Ser. No. 63/438,458, filed on Jan. 11, 2023, the entire contents of which is incorporated herein by reference in its entirety.

### STATEMENT REGARDING GOVERNMENT FUNDING

This invention was made with government support under grant HL141490 awarded by the National Institutes of Health. The government has certain rights in the invention.

### BACKGROUND OF THE INVENTION

### Field of the Invention

The present invention relates generally to fibrosis and more specifically to the use of tissue resident memory T (T_{RM}) cells, such as pulmonary tissue-targeted CD4⁺CD49a⁺ T_{RM} cells, for the treatment of pulmonary fibrosis.

### Background Information

Pulmonary fibrosis is a devastating disease with no known effective treatments to cure, stop or reverse the unremitting, fatal fibrosis. A critical barrier to treating this disease is the lack of understanding of the pathways leading to fibrosis as well as those regulating the resolution of fibrosis. Pulmonary fibrosis is the common final end point for a diverse group of disorders such as chronic hypersensitivity pneumonitis, idiopathic pulmonary fibrosis, silicosis, radiation pneumonitis, and collagen vascular diseases.

Fibrosis is the “dark side” of normal tissue repair that results when the normal wound healing programs go awry. Successful resolution of tissue injury requires not only the activation of effector cells and the marked increase in synthesis and deposition of extracellular matrix (ECM), but also the deactivation of these effector cells and the clearance of excess ECM to allow return to normal lung structure and function. As fibrosis may result from deviations in a number of these pathways, the research disclosed herein sought to elucidate the interplay between immune effectors, inflammatory mediators and fibroproliferation.

Although it is the unregulated fibroproliferation that leads to fibrosis, the mechanisms of fibroblast/myofibroblast recruitment and activation remain poorly understood. While IL-17 and T helper 17 (Th17) cells may promote the inflammation leading to fibrosis, Th1 immune responses in the lung are associated with resolution of inflammation. Accordingly, skewing the lung environment away from the pro-fibrotic Th17 toward a pro-resolution Th1 environment may be important for inhibiting fibrosis.

Supporting the model for local interaction of immune cells with native lung cells is the critical role of tissue resident memory (T_{RM}) cells in the resolution of established lung fibrosis. Fibrosis can stem from immune dysregulation, which itself can direct unremitting fibroproliferation. T_{RM} cells play a critical role in not only directing the anti-pathogen response but also in alleviating inflammation to preserve lung architecture and function. In addition to playing a critical role in the recall response to pathogens, T_{RM}s can mediate protection against tissue specific challenges such as viral, bacterial, and parasitic infections. This protection is achieved by the interaction of T_{RM}s with cells of the adaptive and innate immune systems to collectively coordinate and promote immunity and to dictate the local inflammatory responses. This local response in turn may affect the recruitment, proliferation, and activation of not only immune cells but also resident lung cells.

### SUMMARY OF THE INVENTION

The present invention is based on the seminal discovery that T_{RM} cells are capable of suppressing profibrotic gene expression in proximal cells, inhibiting and even reversing fibrosis.

In one embodiment, the present invention provides a method of treating fibrosis in a subject in need thereof including administering to the subject a composition including a T resident memory (T_{RM}) cell, thereby treating fibrosis.

In one aspect, the T_{RM} cell is CD4⁺. In another aspect, the T_{RM} cell is CD49a⁺. In one aspect, the T_{RM} cell does not express CD103. In another aspect, the T_{RM} cell is CD69⁺, TIM-3⁺, IFNγ⁺, or a combination thereof. In some aspects, IL17a, PD-1, or a combination thereof is downregulated in the T_{RM} cell relative to CD49a−CD4+ T cells. In certain aspects, the T_{RM} cell is derived from the subject. In some aspects, genes in the T_{RM} cell are upregulated following administration of the T_{RM} cell to the subject. In another aspect, the genes encode proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins. In some aspects, the genes are selected from Adamts4, Adamts5, Adamts12, Col1a1, Col1a2, Col4a1, Col4a2, Col4a4, Col5a1, Col8a1, Dcn, Fmod, Hspg2, itgbl1, Lama2, Lamc3, Lum, Postn, Sdc2, Serpina3c, Siglec1, Thbs2, Thbs4, Tll1, or a combination thereof. In some aspects, gene expression is determined by RNA analysis. In one aspect, the fibrosis is in a tissue selected from lung, liver, kidney, heart, muscle, or brain. In some aspects, the fibrosis is in the lung. In some aspects, the lung fibrosis is selected from chronic hypersensitivity pneumonitis, idiopathic pulmonary fibrosis, silicosis, radiation pneumonitis, collagen vascular disease, or a combination thereof. In some aspects, the administering includes intratracheal administration, intrabronchial administration, intranasal administration, nebulization, powder inhalation, intravenous injection, intrapulmonary injection, intraperitoneal, intrathecal, or pulmonary artery infusion. In some aspects, the administering includes intratracheal administration or intranasal administration. In various aspects, the composition includes between about 5×10³ to 5×10⁶ cells. In some aspects, the method further includes repeating the administering after about 1 to 400 days. In some aspects, the method further includes contacting a peripheral blood mononuclear cell (PBMC) with a CD3 antibody, a CD28 antibody, and IL2, thereby generating a T_{RM} cell. In some aspects, the method further includes isolating the T_{RM} cell with a CD4 antibody, a CD49a antibody, or a combination thereof. In another embodiment, the present invention provides a method of increasing a proportion of CD49a⁺CD4⁺ to CD103⁺CD4⁺ cell phenotypes in a tissue of a subject in need thereof including administering to the subject, a composition including a CD49a⁺CD4⁺ T_{RM} cell to the subject, thereby increasing the proportion of CD49a⁺CD4⁺ to CD103⁺CD4⁺ cell phenotypes in a tissue of the subject.

In a further embodiment, the present invention provides a pharmaceutical composition including a CD49a⁺ T resident memory (T_{RM}) cell formulated in a carrier suitable for inhalation or nebulization.

In one aspect, the CD49a⁺ T_{RM} cell is a CD4⁺ cell. In one aspect, the CD49a⁺ T_{RM} cell does not express CD103. In one aspect, the CD49a⁺ T_{RM} cell is a CD69⁺ cell, aTIM-3⁺ cell, an IFNγ⁺ cell, or a combination thereof. In one aspect, genes encoding proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins are upregulated in the CD49a⁺ T_{RM} cell. In one aspect, genes selected from Adamts4, Adamts5, Adamts12, Col1a1, Col1a2, Col4a1, Col4a2, Col4a4, Col5a1, Col8a1, Dcn, Fmod, Hspg2, itgbl1, Lama2, Lamc3, Lum, Postn, Sdc2, Serpina3c, Siglec1, Thbs2, Thbs4, Tll1, or a combination thereof are upregulated in the CD49a⁺ T_{RM} cell. In some aspects, gene expression is determined by RNA analysis. In one aspect, IL17a, PD-1, or a combination thereof is downregulated in the CD49a⁺ T_{RM} cell relative to a CD49a− CD4+ T cell. In one aspect, the pharmaceutical composition includes between about 5×10³ to 5×10⁶ cells.

### BRIEF DESCRIPTION OF THE DRAWINGS

The novel features of the invention are set forthwith particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a set of plots of immune cell and cytokine expression in lungs subjected to buffer (PBS), bleomycin, FluMist®, or Vaccinia treatment. FIG. 1A shows plots of CD49a and CD103 immunostaining data for T cells collected from lungs of mice treated with phosphate buffered saline (PBS, top left), bleomycin (top right), FluMist® (bottom left), and vaccinia virus (bottom right). FIG. 1B shows bar plots summarizing CD49a⁺ and CD103⁺ cell counts in lungs collected from PBS, bleomycin, FluMist®, and vaccinia virus-treated mice. FIG. 1C shows plots with IL17a and IFNγ production in CD49a⁺ and CD103⁺ T cells from lungs of PBS, bleomycin, FluMist®, and vaccinia virus-treated mice.

FIG. 2 is a set of plots of lung physical measurements and immune cell phenotypes in fibrotic and non-fibrotic mice. FIG. 2A shows plots of CD4 and CD45 (top plot) and CD49a and CD69 (bottom plot) immunostaining data for T cells collected from lungs of vaccinated mice. FIG. 2B shows plots summarizing pulmonary function testing resistance (upper left plot), compliance (upper right plot), and diffusion capacity (lower plot) measurements on non-fibrotic (PBS-treated) mice, fibrotic mice administered CD4⁺CD49a⁻ T_{RM} cells, and fibrotic mice administered CD4⁺CD49a⁺ T_{RM} cells. FIG. 2C shows bar plots of collagen 1 (left plot) and collagen 3 (right plot) RNA expression in lungs of non-fibrotic mice, fibrotic mice administered CD4⁺CD49a⁻ T_{RM} cells, and fibrotic mice administered CD4⁺CD49a⁺ T_{RM} cells.

FIG. 3 is a set of plots of physical measurements and images of fibrotic and non-fibrotic lungs. FIG. 3A shows plots summarizing changes in pulmonary function testing resistance (upper left plot), compliance (upper right plot), and diffusion capacity (lower plot) over a 38 day span in non-fibrotic (PBS-treated) mice, fibrotic mice administered CD4⁺CD49a⁻ T_{RM} cells, and fibrotic mice administered CD4⁺CD49a⁺ T_{RM} cells. FIG. 3B shows histological staining images of a non-fibrotic mouse lung (top left), a fibrotic mouse lung (top right), a fibrotic mouse lung treated with CD4⁺CD49a⁻ T_{RM} cells (bottom left), and a fibrotic mouse lung treated with CD4⁺CD49a⁺ T_{RM} cells (bottom right).

FIG. 4 is a set of plots of expression and phenotype data for blood derived immune cells and lung-derived fibroblasts. FIG. 4A shows a plot summarizing CD49a expression in CD49a⁺ and CD49a⁻ CD4⁺ T_{RM} cells. FIG. 4B shows plots summarizing TNFa (top left), granzyme B (top right), IFN gamma (bottom left), and IL17a (bottom right) levels in CD49a⁺ and CD49a⁻ CD4⁺ T_{RM} cells. FIG. 4C shows bar plots of relative collagen RNA expression (upper left), relative smooth muscle actin RNA expression (upper right), and relative elastin RNA expression (lower) in fibroblasts from normal or fibrotic lungs cocultured with CD4⁺CD49a⁺ or CD4⁺CD49a⁻ T_{RM} cells. FIG. 4D shows plots of TNFa (left plot) and IFN gamma (right plot) levels in fibroblast, CD4⁺ T_{RM} cell cocultures. FIG. 4E shows plots summarizing live/dead dye flow cytometry measurements on fibroblasts (top), cocultures with fibroblasts and CD4⁺CD49a⁻ T_{RM} cells (middle), and fibroblasts and CD4⁺CD49a⁺ T_{RM} cells (bottom).

FIG. 5 is a set of plots of TRM gene expression. FIG. 5A is a plot of differential gene expression of CD49a⁺ CD4⁺ TRM versus CD49a− CD4⁺ TRM. FIG. 5B is a plot of STRING analyses highlighting upregulated genes in CD4⁺CD49a⁺ T_{RM} cells.

FIG. 6 is a set of plots and images of CD4, CD49a, and CD103a expression in TRMs. FIG. 6A is a set of plots of processed scRNAseq data of CD4⁺CD49a⁻ and CD4⁺CD103a⁺ TRMs from fibrotic human lungs. FIG. 6B is a set of images of biopsy samples from normal (left) and fibrotic (right) lungs stained for CD4 and CD103 (top) and CD4 and CD49a (bottom).

### DETAILED DESCRIPTION OF THE INVENTION

The present invention is based on the seminal discovery that T_{RM} cells are capable of suppressing profibrotic gene expression in proximal cells, inhibiting and even reversing fibrosis.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

The present disclosure provides T_{RM} cell compositions and therapies which inhibit and reverse fibrosis. As used herein, the term “tissue resident memory T cell” (T_{RM} cell) denotes lymphocyte cells capable of occupying nonlymphoid tissues without recirculating. T_{RM} cells often express lectin and integrin repertoires which phenotypically distinguish them from other memory T cells and which can facilitate tissue-targeting specificity.

Contrasting prevailing theories which recognize a “point of no return” for fibrosis, the results disclosed herein demonstrate that fibrosis (for example increased extracellular matrix (ECM) deposition), fibroproliferation, and chronic inflammation may not only be arrested but also reversed by promoting normal wound repair pathways. In particular, it was determined herein that T_{RM} cells can promote immune responses which regulate fibrosis and re-establish tissue homeostasis, and furthermore that T_{RM} cells can diminish profibrotic gene expression in proximal cells and tissues. Furthermore, it is disclosed herein that adoptive transfer of CD49a⁺ T_{RM} cells is sufficient to reverse established murine fibrosis, even in the absence of vaccination.

Although some memory T cells circulate in the blood and amongst secondary lymphoid organs as effector memory cells (Tem), memory T cells also take up permanent residence in specific tissue compartments. These tissue resident memory (T_{RM}) cells, generated in response to site specific infections in lungs, skin, etc., are non-migratory and specifically maintained in these tissues. In particular, T_{RM} cells play essential roles in the recall response to mediate protection both against tissue specific and non-specific challenges such as viral, bacterial, and parasitic infections. For example, in the lungs both CD4⁺ and CD8⁺ T_{RM} are important for protection against influenza. Interestingly, although both injectable inactivated influenza vaccine (IIV) and intranasal live attenuated influenza vaccine (LAIV) generate neutralizing strain-specific antibodies, only the intranasally administered LAIV generates lung localized, virus specific T cell responses similar to what is generated with influenza infection. More importantly, only the intranasal LAIV generates TRM that mediate cross-strain protection, independent of circulating T cells and neutralizing antibodies. Thus, intranasal LAIV generation of lung T_{RM} cells not only protects from future infection to the specific vaccinated viral strain but also provides heterosubtypic protection to non-vaccine viral strains.

It is further disclosed herein that vaccines such as vaccinia and influenza vaccines (e.g., FluMist®) induce T_{RM} cells that reverse fibrosis. The vaccine induced T_{RM} cells CD4⁺CD49a⁺, were isolated and were shown to be sufficient to reverse established fibrosis. In fact, adoptive cellular therapy results disclosed herein demonstrate that intratracheal administration of CD4⁺CD49a⁺ T cells into established fibrosis reverses the fibrosis histologically by promoting a decrease in collagen levels, without the need for vaccination. In addition, the paucity of these cells in histologic samples from patients with IPF as well as co-culture of in vitro derived CD4⁺CD49a⁺ T_{RM} cells with human IPF fibroblasts results in a down regulation of IPF fibroblast collagen production. The discovery that CD49a⁺ T cells are relatively absent in IPF lungs support a strategy of augmenting CD49a⁺ TRMs, via adoptive transfer as a means of promoting resolution of fibrosis and normal healing.

The development of fibrosis is a complex process that involves multiple cell types in the lung. Indeed, numerous studies, both mouse and human, have implicated not only fibroblasts, epithelial and immune cells but also a host of cytokines, chemokines and transcription factors as playing essential roles in the excess accumulation of ECM characteristic of lung fibrosis. Without being bound by theory, the tissue-specific adoptively transferred T_{RM}s disclosed herein may interact with host immune cells to promote normal resolution of lung injury. In particular, the bulk RNA sequencing data disclosed herein suggest that CD49a⁺ T_{RM} cells have broad-ranging effects on processes that promote and maintain fibrosis. STRING analysis showed that CD49a⁺ T_{RM} cells upregulate distinct molecular pathways involved in collagen binding and signaling, extracellular matrix organization, metalloproteinases and growth factor and glycosaminoglycan binding. Thus, CD49a⁺ T_{RM} cells reverse established fibrosis by promoting remodeling of the ECM, perhaps resetting the imbalance of injury to wound healing.

Notably, the studies disclosed herein utilized mouse models in which chronic fibrosis was induced with IP injections of bleomycin over four weeks leading to progressive low-grade immune cell infiltration, collagen deposition and fibrotic changes in the mouse lungs at 72⁺ days. This model differs from an acute IT bleomycin model because it never causes an acute inflammatory phase but rather promotes sub-acute inflammation that progresses to fibrosis over months. This may be in part be due to the fact that the standard intratracheal bleomycin model causes acute lung injury that heals with variable fibrosis. This model is often not progressive beyond the first few weeks and has been demonstrated to be reversible without any intervention over time. Indeed, most drug interventions in this model are tested during this acute/subacute injury and may indeed only reflect accelerated resolution of lung injury thus preventing fibrosis. As an acute inflammatory stage is often unrecognized in humans and the lung fibrosis progresses slowly and continuously over time, the mouse model utilized in the studies disclosed herein more closely approximate human disease, and enabled identification of treatments that alleviate and reverse pulmonary fibrosis.

Leveraging these discoveries, the present invention provides therapeutic compositions of T_{RM} cells as well as associated methods of use for treating fibrosis. Increasing an abundance of T_{RM} cells in fibrotic tissue can not only halt the progression of fibrosis, but can activate tissue repair pathways which reverse its effects. Furthermore, the T_{RM} cells can reduce inflammation, which is a driver of many fibrotic pathways. Such therapeutic T_{RM} cell responses can be generated by autologous or allogenic T_{RM} cell administration or by T_{RM} cell induction (e.g., with a vaccine). While fibrosis has previously been viewed as an irremediable condition, the present invention provides compositions and methods for inhibiting and reversing this condition.

In one embodiment, the present invention provides a method of treating fibrosis in a subject in need thereof including administering to the subject a composition including a T resident memory (T_{RM}) cell, thereby treating fibrosis.

As used herein, the term “fibrosis” refers to fibrous tissue formation beyond levels observed in healthy tissues. Fibrosis is often a reparative response to injury or damage and can refer to the connective tissue deposition that occurs as part of normal healing or to the excess tissue deposition that occurs as a pathological process. Fibrosis can include extracellular matrix (ECM) and collagen deposition at levels which diminish elasticity and inhibit normal tissue function. Fibrosis can also denote overaccumulation or overactivity of fibroblasts in an organ or tissue. When fibrosis occurs in response to injury, the term “scarring” is used. By treating fibrosis, it is meant that the methods described herein diminish fibrotic processes such as collagen deposition or profibrotic fibroblast activity, or reverse effects of fibrosis, such as collagen density or tissue hardening.

As used herein, the term “fibroblast” can denote a connective tissue cell which forms extracellular matrices (ECMs) and collagen.

The term “subject” as used herein refers to any individual or patient to which the disclosed methods are performed, to whom the disclosed compositions are administered, or from whom a biological material (e.g., a tissue sample, a cell, or a biofluid) is obtained. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In some aspects, administering includes intratracheal administration, intrabronchial administration, intranasal administration, nebulization, powder inhalation, intravenous injection, intrapulmonary injection, intraperitoneal, intrathecal, or pulmonary artery infusion. In some cases, the administering includes intratracheal or intranasal administration.

In certain aspects, the methods treat fibrosis and increase a proportion of CD49a⁺CD4⁺ to CD103⁺CD4⁺ cell phenotypes in a tissue of the subject. In some such cases, the tissue is pulmonary tissue. The composition may be any composition disclosed herein (e.g., any pharmaceutical composition disclosed herein). The T_{RM} cell can be autologous (i.e., derived from the subject), allogenic (i.e., derived from another subject of the same species), or a combination thereof.

In some aspects, the T_{RM} cell is CD4⁺ (i.e., expresses CD4). In many such cases, the T_{RM} cell is CD4⁺CD8⁻. As disclosed herein, CD4⁺ (e.g., T helper) T_{RM} cells can affect localized changes in gene expression which diminish fibrotic activity, for example by inhibiting fibroblast-mediated collagen deposition, and can promote wound repair pathways which inhibit and reverse fibrosis.

The T_{RM} cell can also express an integrin or subunit thereof (e.g., CD49a). While integrins are well known extracellular matrix binders, a surprising discovery herein is that the expression of certain integrins can contribute pro-fibrotic resolution phenotypes. In some aspects, the T_{RM} cell is CD49a⁺. In particular cases, the T_{RM} cell is CD4⁺CD49a⁺. In some aspects, the T_{RM} cell does not express CD103 (e.g., is CD4⁺CD49a⁺CD103⁻). In some aspects, the T_{RM} cell is CD69⁺, TIM-3⁺, IFNγ⁺, or a combination thereof.

The T_{RM} cell can be autologous (i.e., derived from the subject to which they are administered) or allogenic (i.e., derived from and delivered to different subjects). In some cases, the T_{RM} cell is derived from the subject.

In certain aspects, genes in the T_{RM} cell are upregulated following administration of the T_{RM} cell to the subject. In some aspects, the genes encode proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins. In some aspects, the genes are selected from Adamts4, Adamts5, Adamts12, Col1a1, Col1a2, Col4a1, Col4a2, Col4a4, Col5a1, Col8a1, Dcn, Fmod, Hspg2, itgbl1, Lama2, Lamc3, Lum, Postn, Sdc2, Serpina3c, Siglec1, Thbs2, Thbs4, Tll1, or a combination thereof. The gene expression may be determined by any technique known in the art. In some aspects, the gene expression is determined by RNA analysis.

In some aspects, a proinflammatory marker is downregulated in the T_{RM} cell relative to a CD49a−CD4+ T cell. For example, in some aspects, IL17a is downregulated in the T_{RM} cell relative to CD49a−CD4+ T cells. IL17a is a proinflammatory cytokine associated with numerous chronic pathologies, including fibrosis. Diminishing IL17a in tissue can not only diminish fibrotic pathogenesis but can promote pro-resolution environments conducive to reversing fibrosis. In some aspects, PD-1 (programmed cell death protein 1) is downregulated in the T_{RM} cell relative to a CD49a−CD4+ T cell. PD-1 is an immunosuppressive surface protein expressed by some B and T cells which can promote the progression of idiopathic pulmonary fibrosis. In some cases, IL17a and PD-1 are downregulated in the T_{RM} cell relative to CD49a−CD4+ T cells.

The administration can provide systemic or localized delivery of the T_{RM} cell. In many cases, the pharmaceutical composition is formulated for localized delivery. Following systemic or localized delivery (e.g., by intravenous administration), the T_{RM} cell may localize to a particular tissue based on its phenotype. As non-limiting examples, CD4⁺CD49a⁺, CD4⁺CD69⁺, and CD8⁺CXCR6⁺ T_{RM} cells may separately localize to pulmonary, dermal, and hepatic tissues following systemic administration. In some cases, the T_{RM} cell migrates to a target organ or tissue following localized administration. In some cases, T_{RM} cell expresses a receptor which prevents its egress from a particular tissue or interstitial space (e.g., a particular integrin or a subunit thereof). For example, a CD49a⁺ TRM may be retained within a lung following transit into the lung upon tracheal administration. In some cases, the T_{RM} cell is configured to localize to a lung, liver, kidney, heart, muscle, or brain following administration. In some cases, the T_{RM} cell is configured to localize to pulmonary tissue following intratracheal or intranasal administration.

Aspects of the present disclosure utilize T_{RM} cell localization to mitigate fibrosis in a tissue, organ, or location-specific manner. For example, a systemically administered T_{RM} cell may localize to a lung and inhibit pulmonary fibrosis.

The invention provides methods for treating fibrosis in a subject. In one aspect, the fibrosis is in a tissue selected from the group consisting of lung, liver, kidney, heart, muscle, or brain. In some aspects, the fibrosis is lung (i.e., pulmonary) fibrosis. In particular aspects, the lung fibrosis is selected from chronic hypersensitivity pneumonitis, idiopathic pulmonary fibrosis, silicosis, radiation pneumonitis, collagen vascular disease, or a combination thereof.

In some aspects, the composition has between about 5×10² to 5×10⁷ cells. In some aspects, the composition has between about 5×10³ to 5×10⁶ cells. In some aspects, the composition has between about 5×10³ and 5×10⁵, between about 5×10⁴ and 5×10⁶ cells or between about 10⁵ and 5×10⁶ cells. In some aspects, at least about 50%, at least about 60%, at least about 70%, at least about 80%, at least about 85%, at least about 90%, at least about 95%, at least about 98%, or at least about 99% of the cells are T_{RM} cells (e.g., instances of the T_{RM} cell as described further herein).

The subject can be administered a single dose or multiple doses of the composition. In some aspects, the doses are identical, or differ in form (e.g., intravenous versus intratracheal), excipients, and number and types of T_{RM} cells. In some aspects, identical doses of the composition are administered to the subject at least 2, 3, 4, 5, 6, 7, 8, 9, 10, or more times. In some aspects, the subject is administered doses of the composition until their fibrosis is halted or ameliorated. For example, the subject may be administered doses of the composition in regular intervals until they surpass predetermined system resistance (Rrs), compliance (Crs), and/or elastance (Ers) thresholds in pulmonary mechanics tests. In some aspects, the method includes repeating the administering after about 1 to 400 days. For example, a single dose of the composition is administered to the subject on a daily, weekly, biweekly, monthly, bimonthly, semiannual, or annual basis.

In a further embodiment, the present invention provides a method of increasing a proportion of CD49a⁺CD4⁺ to CD103⁺CD4⁺ cell phenotypes in a tissue of a subject in need thereof by administering to the subject a composition which includes a CD49a⁺CD4⁺ T_{RM} cell, thereby increasing the proportion of CD49a⁺CD4⁺ to CD103⁺CD4⁺ cell phenotypes in a tissue of the subject.

Aspects of the present invention provide methods for generating a T_{RM} cell. The T_{RM} cell can be included in a composition and utilized in a method (e.g., administered) as disclosed herein. For example, in an embodiment, the present invention provides a method of treating fibrosis in a subject in need thereof including administering to the subject a composition including a T resident memory (T_{RM}) cell, thereby treating the fibrosis, wherein prior to administering the cells, the method further includes generating the T_{RM} cell.

In some aspects, the generating the T_{RM} cell includes contacting a peripheral blood mononuclear cell (PBMC) with a CD3 antibody, a CD28 antibody, and IL2.

In certain aspects, the method further includes isolating the T_{RM} cell with a CD4 antibody, a CD49a antibody, or a combination thereof. In particular aspects, the isolating yields a population of cells of which at least about 50%, at least about 60%, at least about 70%, at least about 80%, at least about 85%, at least about 90%, at least about 95%, at least about 98%, or at least about 99% are T_{RM} cells. In some aspects, the isolating yields a population of cells of which at least about 50%, at least about 60%, at least about 70%, at least about 80%, at least about 85%, at least about 90%, at least about 95%, at least about 98%, or at least about 99% are CD4⁺CD49a⁺. In some aspects, the isolating yields a population of cells of which at least about 50%, at least about 60%, at least about 70%, at least about 80%, at least about 85%, at least about 90%, at least about 95%, at least about 98%, or at least about 99% are CD4⁺CD49a⁺CD103⁻.

In an embodiment, the present disclosure provides a pharmaceutical composition including a T resident memory (T_{RM}) cell. In various aspects, the T_{RM} cell is active in treating, preventing, or inhibiting fibrosis. The pharmaceutical composition can include a plurality of immune cells, of which at least about 50%, at least about 60%, at least about 70%, at least about 80%, at least about 85%, at least about 90%, at least about 95%, at least about 98%, or at least about 99% are T_{RM} cells. For many of the embodiments disclosed herein, the T_{RM} cell is a living T_{RM} cell. Upon administration, the T_{RM} cell localize to a target tissue or organ, thereby affecting a localized antifibrotic response. For example, the T_{RM} cell express a receptor which prevents egress from a target organ, tissue, or interstitial location.

In certain aspects, the T_{RM} cells are CD49a⁺. CD49a is a subunit of the collagen- and laminin-binding integrin VLA-1 which localizes cells to ECM and which can alter immunomodulatory activity, neutrophil recruitment, and cytokine repertoire. CD49a⁺ T_{RM} cells can also inhibit profibrotic gene expression, rendering them effective for preventing and treating fibrosis. In some cases, at least about 50%, at least about 60%, at least about 70%, at least about 80%, at least about 85%, at least about 90%, at least about 95%, at least about 98%, or at least about 99% of T_{RM} cells in the pharmaceutical composition are CD49a⁺.

The pharmaceutical composition can be formulated for a variety of forms of administration, including enteral, topical or parenteral. As nonlimiting examples, the pharmaceutical composition can be formulated for intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal, intrasternal, oral, sublingual buccal, rectal, vaginal, nasal, ocular, infusion, inhalation, or nebulization-based administration. In some cases, the pharmaceutical composition is configured for intratracheal administration, intrabronchial administration, intranasal administration, nebulization, powder inhalation, intravenous injection, intrapulmonary injection, intraperitoneal, intrathecal, or pulmonary artery infusion.

In certain aspects, the pharmaceutical composition is formulated in a carrier suitable for inhalation or nebulization. An inhalable or nebulizable composition as disclosed herein can be formulated as a liquid or dry powder amenable for use with any known spray, inhaler or nebulization system in the art, including pressure-driven aerosol nebulizers, ultrasonic nebulizers, and nasal sprays. Nonlimiting examples of carriers include solvents such as water, ethanol, ethylene glycol, and propylene glycol, powders such as dextran, glucose, and corn starch, and micellular and lipid vehicles such as liposomes. In some aspects, the pharmaceutical composition is configured for intratracheal or intranasal administration (for example through nebulization).

As it was demonstrated herein that certain CD4⁺ T_{RM} cells inhibit and reverse fibrosis, in many aspects, the T_{RM} cell is CD4⁺. In some aspects, at least about 50%, at least about 60%, at least about 70%, at least about 80%, at least about 85%, at least about 90%, at least about 95%, at least about 98%, or at least about 99% of T_{RM} cells in the pharmaceutical composition are CD4⁺. In some cases, the T_{RM} cell is CD4⁺CD49a⁺. For example, in some cases, at least about 50%, at least about 60%, at least about 70%, at least about 80%, at least about 85%, at least about 90%, at least about 95%, at least about 98%, or at least about 99% of T_{RM} cells in the pharmaceutical composition are CD4⁺CD49a⁺.

In some aspects, the T_{RM} cell does not express CD103. For some cells, CD103 expression can promote pathogenic, profibrotic phenotypes, and can therefore be inimical during fibrosis treatment. Accordingly, in some cases, the T_{RM} cell is CD4⁺CD103⁻. In some cases, the T_{RM} cell is CD49a⁺CD103⁻. In some cases, the T_{RM} cell is CD4⁺CD49a⁺CD103⁻. In some cases, less than about 50%, less than about 40%, less than about 30%, less than about 20%, less than about 15%, less than about 10%, less than about 5%, less than about 2%, or less than about 1% of T_{RM} cells in the composition express CD103.

In some aspects, genes encoding proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins are upregulated in the T_{RM} cell. In some aspects, the genes are selected from Adamts4, Adamts5, Adamts12, Col1a1, Col1a2, Col4a1, Col4a2, Col4a4, Col5a1, Col8a1, Den, Fmod, Hspg2, itgbl1, Lama2, Lamc3, Lum, Postn, Sdc2, Serpina3c, Siglec1, Thbs2, Thbs4, Tll1, or a combination thereof.

In some aspects, IFNγ is upregulated in the T_{RM} cell relative to CD49a−CD4+ T cells. In other aspects, IL17a, PD-1, or a combination thereof is downregulated in the T_{RM} cell relative to CD49a−CD4+ T cells (e.g., CD49a−CD4+ T cells from the same subject from the same preparation from which the T_{RM} cell was obtained). In some aspects, gene expression is determined by RNA analysis, example with RNA-seq or with a gene expression microarray.

In some cases, the T_{RM} cell is autologous (i.e., derived from the subject to which they are administered). In some cases, the T_{RM} cell is allogenic (i.e., administered to a different subject than the cells were derived from). In some cases, the T_{RM} cell is obtained from peripheral blood, for example from a buffy coat or leukopak. In some cases, the T_{RM} cell is generated from a peripheral blood mononuclear cell (PBMC). In some cases, the T_{RM} cell is generated from a leukocyte.

In some cases, the pharmaceutical composition does not include proinflammatory T helper 17 (Th17) cells. In some cases, less than about 50%, less than about 40%, less than about 30%, less than about 20%, less than about 15%, less than about 10%, less than about 5%, less than about 2%, or less than about 1% of cells in the composition are Th17 cells.

In some aspects, the pharmaceutical composition has a therapeutically effective amount of the T_{RM} cell, for example between about 5×10² to 5×10⁷ cells. In some cases, the pharmaceutical composition has between about 5×10³ to 5×10⁶ cells. In some cases, the pharmaceutical composition has between about 5×10³ and 5×10⁵, between about 5×10⁴ and 5×10⁶ cells or between about 10⁵ and 5×10⁶ cells. In some cases, at least about 50%, at least about 60%, at least about 70%, at least about 80%, at least about 85%, at least about 90%, at least about 95%, at least about 98%, or at least about 99% of the cells are T_{RM} cells (e.g., CD4⁺CD49a⁺CD103⁻ T_{RM} cells).

The term “effective amount” of an active agent refers an amount that is non-toxic to a subject or a majority or normal cells but is an amount of the active agent that is sufficient to provide a desired effect (e.g., treatment of a skeletal muscle disorder, metabolic disorder, blood disorder, or cancer). This amount may vary from subject to subject, depending on the species, age, and physical condition of the subject, the severity of the disease that is being treated, the particular conjugate, or more specifically, the particular active agent used, its mode of administration, and the like. Therefore, it is difficult to generalize an exact “effective amount,” yet, a suitable effective amount may be determined by one of ordinary skill in the art.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., the prevention or amelioration of fibrosis). Such an amount should be sufficient to inhibit or treat fibrosis, and can be determined as described herein.

By “pharmaceutical composition” it is meant that the cells described herein are formulated with a “pharmaceutically acceptable” carrier, diluent or excipient that is compatible with the other ingredients of the composition and not deleterious to the recipient thereof, nor to the activity of the active ingredient (e.g., the T_{RM} cell). Pharmaceutically acceptable carriers, excipients or stabilizers are well known in the art, and include those disclosed in Remington's Pharmaceutical Sciences, 16th edition, Osol, A. Ed. (1980). Pharmaceutically acceptable carriers, excipients, or stabilizers are typically nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carriers include, but are not limited to, liposomes, nanoparticles, microparticles, polysaccharides, hydrogels (e.g., alginates), ointments, micelles, microspheres, creams, emulsions, and gels. Examples of excipients include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluents include, but are not limited to, water, alcohol, saline solution, glycol, mineral oil, and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

In certain embodiments, the compositions disclosed herein are formulated with additional agents that promote entry into the desired organ, interstitial space, or tissue. Such additional agents can include micelles, liposomes, and dendrimers.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, and nebulizable mediums.

The term “pharmaceutically acceptable salts” refers to physiologically and pharmaceutically acceptable salts of the compounds of the invention, e.g., salts that retain the desired biological activity of the parent compound and do not impart undesired toxicological effects thereto.

Presented below are examples discussing the peptides and multiepitope peptides described herein, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Materials and Methods

### Mice

C57BL/6 were purchased from The Jackson Laboratory. 8 to 10-week-old female and male mice were utilized for experiments.

### Reagents

Mice were inoculated with a modified vaccinia ankara virus which contained the full-length ovalbumin protein but lacked lytic ability. Quadrivalent FluMist® was purchased from AstraZeneca (Wilmington, DE). Bleomycin was purchased from App Pharmaceuticals (Schumburg, IL). PMA and Ionomycin were purchased from Sigma-Aldrich (St. Louis, MO). Flow cytometry reagents were purchased from BD Biosciences (Franklin Lakes, NJ). Antibodies utilized were from Biolegend (San Diego, CA).

### Cell Lines

Age matched normal and IPF lung fibroblasts were purchased from BioIVT (Westbury, NY). Fibroblasts were maintained in DMEM supplemented with 10% FBS. For all coculture experiments fibroblasts were used between passages 3-6. To generate human CD49a+ and CD49a− CD4+ T cells, lymphocytes were isolated from buffy coats of normal donor leukopaks. Bulk lymphocytes were stimulated with anti-CD3/anti-CD28 (Miltenyi Biotech) in RPMI media supplemented with 10% FBS, antibiotic, and glutamine. Two days later human IL2 (Peprotech, East Windsor, NJ), was added at 10 pg/mL. Cultures were maintained for 4 weeks with addition of media as needed and addition of IL2 every 7 days.

### Coculture Experiments

Fibroblasts were harvested and plated at a density of 50,000 per well of a 24 well transwell plate (Corning, Corning, NY.) Fibroblasts were allowed to adhere for 6 hours before addition of T cells. Live human lymphocytes in culture were isolated by Ficoll-Paque (Fisher Scientific, Waltham, MA). CD4+ T cells were then isolated by negative magnetic isolation (Biolegend). CD49a+ and CD49a− cells were isolated by magnetic isolation using Anti-CD49a purified antibody (Biolegend) and anti-mouse IgG microbeads (Miltenyi Biotech). Purified CD4 T cells were added into transwells of 24 well plates in DMEM media supplemented with 10 pg/mL human IL2. Cocultures were incubated overnight, followed by addition of 10 pg/mL TGFβ (Peprotech). 24 hours later fibroblast RNA was harvested.

### Flow Cytometry

All Flow cytometry analysis was performed on a BD FACSCelesta (BD Biosciences) and analyzed using FlowJo software (TreeStar Inc, Ashland, OR).

### Pulmonary Fibrosis Model

Mice were injected intraperitoneally with 0.8 units bleomycin on days 0, 3, 7, 10, 14, 21, and 28 to induce pulmonary fibrosis. For some experiments mice received bleomycin intratracheally at a dose of 0.018 units in 25 ul.

### Vaccination

Vaccinia vaccine was administered intranasally at a dose of 2 million pfu per mouse. Quadrivalent FluMist® was administered intranasally at a dose of 105.5-6.5 fluorescent focus units of live attenuated influenza virus reassortants of each of the four strains: A/Victoria/1/2020 (H1N1) (A/Victoria/2570/2019 (H1N1) pdm09-like virus), A/Norway/16606/2021 (H3N2) (A/Darwin/9/2021 (H3N2)-like virus), B/Phuket/3073/2013 (Yamagata lineage), and B/Austria/1359417/2021 (Victoria lineage).

### ELISA

TNFα and IFNγ ELISA were purchased from ebioscience (San Diego, CA), and performed according to manufacturer's instructions.

### Histology

Lungs were inflated to atmospheric pressure with formalin and sectioned and stained for H&E and Masson's trichrome, and in particular to 27 cm H₂O with 10% neutral-buffered formalin, sectioned and stained for H&E and Masson's trichrome according to previously established procedures (Limjunyawong, et al. J Vis Exp, e52964 (2015)). Samples were analyzed by microscope at 40× magnification.

### RNA Extraction and Real Time PCR

Total lung RNA was extracted using TRIzol reagent (Invitrogen, Carlsbad, CA) and reversed transcribed by Reliance (Bio-Rad Laboratories, Hercules, CA) as per the manufacturer's protocol. Real-time PCR was performed on an Applied Biosystems 7300 PCR machine using Applied Biosystems reagents (Carlsbad, CA) and normalized to 18s rRNA. Values were calculated using the delta Ct method in reference to control samples for each primer. All primers and probe sets used were purchased from Applied Biosystems (Carlsbad, CA).

### In Vivo Antibody Labeling and Sorting

For in vivo antibody labeling, mice were injected i.v. with 2.5 μg PE-conjugated anti-CD4 antibody (clone RM4-5), and after 10 minutes, lungs were isolated, rinsed in PBS, and digested in a mixture of 3 ug/mL Collagenase and Dnase I for 45 minutes at 37° C. Cells were filtered into single cell suspensions, and CD4+ T cells were positively selected by magnetic isolation (Miltenyi Biotech (Auburn, CA). Isolated lymphocytes were then stained in vitro with a different, noncompeting clone of APC-anti-CD4 (clone RM4-4), along with antibodies to other surface markers with fluorochrome-conjugated antibodies. Stained cells were sorted using a BD FacsAria (BD Biosciences, Franklin Lakes, NJ). For T_{RM} transfer experiments, sorted T cells were resuspended in PBS at 1×106 per ml and mice received 50 ul intratracheally.

### Diffusion Factor for CO Measurement

To assess the efficiency of gas exchange in the lungs following bleomycin-induced injury, measurement of the diffusion factor for CO (DFCO) was performed as described previously. Briefly, mice were anesthetized with a mixture of ketamine (100 mg/kg)/xylazine (15 mg/kg) via i.p. injection. Once sedated, mice were intubated with a 20-gauge IV angiocatheter. Mouse lungs were quickly inflated with a 0.8 ml gas mixture (0.5% neon, 1% CO and 98.5% air). After a 9-second breath hold, 0.8 m of gas was quickly withdrawn from the lung and diluted to 2 ml with room air. The neon and CO concentrations in the diluted air were measured by gas chromatography (INFICON, Model 3000A) to assess DFCO. The dilution to 2 ml was needed, since the gas chromatograph required a minimal sample size of 1 ml.

### Pulmonary Mechanics Measurements

After DFCO assessment, mice were connected to a flexi-Vent ventilator (SCIREQ) and ventilated with a tidal volume of 0.2 ml of 100% oxygen at a rate of 150 Hz. with a positive end-expiratory pressure (PEEP) of 3 cmH2O. Mice were subjected to deep inspiration at 30 cmH2O for 5 seconds and returned to normal ventilation for 1 minute. Baseline measurements of respiratory system resistance (Rrs), compliance (Crs) and elastance (Ers) were measured using forced oscillation technique via the SnapShot perturbation, a single 2.5-Hz sinusoidal waveform which is fit to the single compartment model via linear regression. Following measurements angiocatheters were removed and mice were observed until they woke up from anesthesia.

### Statistical Analysis

All experiments were performed in biological triplicate and results represent the mean standard deviation. All experiments were replicated at least three times. Statistical analysis was performed using either 1-way ANOVA followed by Tukey's test or a paired Student's t-test; P<0.05 was considered statistically significant.

### RNA Sequence Analysis

Wild type mice were vaccinated intranasally with FluMist and 4 weeks later in vivo labeled CD49a⁺ CD4⁺ lung TRM and CD49a⁻CD4⁺ lung TRM were sorted into Trizol. RNA was isolated and subjected to RNA seq analysis. Alignments were performed using Hisat2, and differential gene expression was performed using DESeq2 and BiomaRt. The top 200 most differentially expressed genes in CD49a⁺CD4⁺ lung TRMs were submitted to STRING analysis of known and predicted protein-protein interactions. Clustering of upregulated proteins resulting in a cluster which contained 26 upregulated genes with a small PPI enrichment p-value (<10e-16), indicating a high likelihood that the proteins are biologically connected.

### Analysis of RNA Sequence Data

Processed scRNAseq data for human lungs with fibrosis were obtained from the Gene Expression Omnibus database (accession number GSE135893) (Adams et al. Sci Adv 6, eaba1983 (2020)). Annotated T cells were clustered using the R software package Seurat (v.4.2). CD49a⁺ TRM were identified by CD4 and ITGA1 (CD49a) expression (log-transformed expression levels>0.5). CD103a⁺ TRM were identified by CD4 and ITGAE (CD103) expression. TRMs were plotted as % of annotated T cells in IPF (n=28) and non-fibrotic control (n=14) lungs. Lungs with fewer than 100 T cells were excluded.

### Immunofluorescence Staining

Formalin-fixed, paraffin-embedded (FFPE) human IPF and Normal tissue slides were incubated with primary antibody to CD4, CD49a, and CD103 in 5% goat serum, 0.5% BSA, in PBS at 4° C. overnight. Images were collected at ×20 magnification using a Nikon Eclipse 80i microscope and Nikon DS-fil camera.

### Example 2

### CD49a and CD103 Expression Differentiates Populations of Lung CD4+ T_{RM} Cells

This example covers the identification of T_{RM} phenotypes which are protective against lung fibrosis. Mice were first administered PBS, quadrivalent FluMist®, or vaccinia virus to induce T_{RM} generation. To analyze T_{RM} generation during the development of fibrosis, a group of mice were treated with further subjected to intratracheal bleomycin treatments. After twenty-eight days, lung tissue resident CD4+ T cells were intravenously labeled and isolated with CD4 antibodies. The mice were sacrificed, and their lungs were removed, processed to form single cell suspensions, and stained for CD4 and the surface markers CD49a and CD103. CD49a+ and CD103+ CD4+ lung T_{RM} were sorted and stimulated for 4 hours to perform intracellular cytokine staining (ICS).

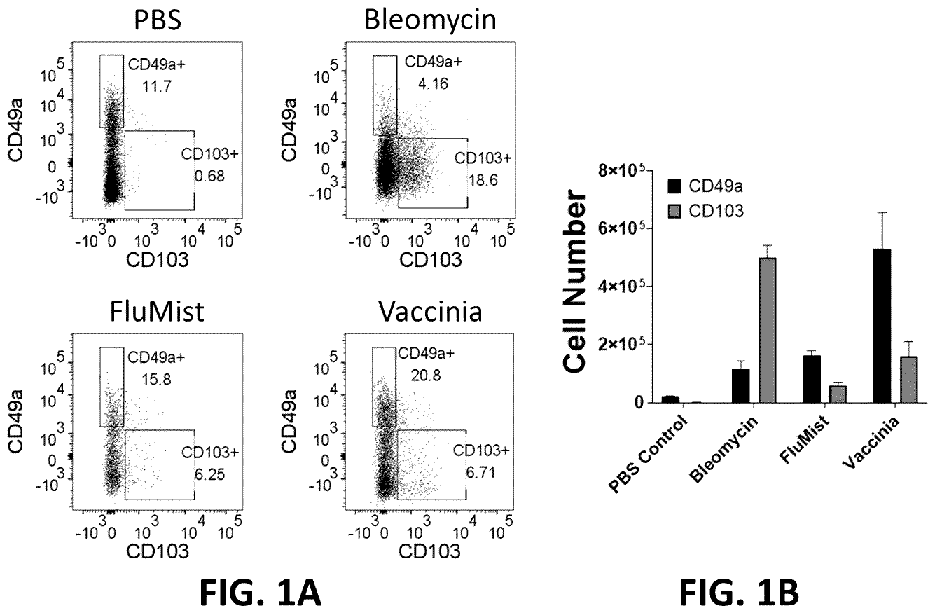

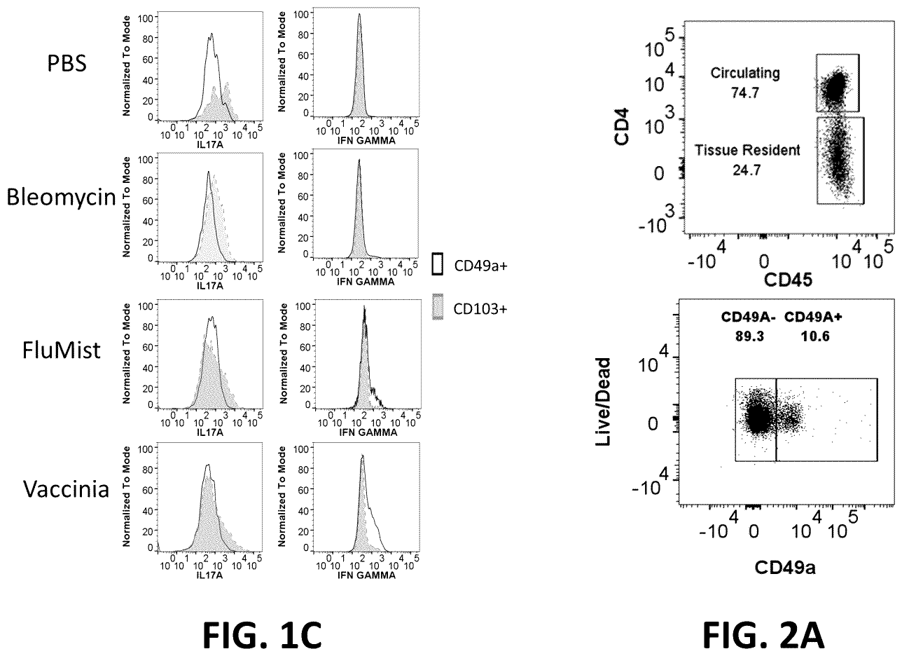

The results of these analyses are shown in FIGS. 1A-C. T_{RM} from bleomycin treated mice were predominantly CD103+, whereas T_{RM} from vaccinated mice were predominantly CD49a+ (FIGS. 1A-B). PBS treated mice also had predominantly CD49a+ T_{RM}, however the percentages and total numbers of these cells were significantly lower than vaccinated mice. ICS analysis of sorted CD4+ T_{RM} demonstrated increased expression of IL17a and minimal expression of IFNγ by CD103+ T_{RM} regardless of how mice were treated (FIG. 1C). Intracellular cytokine staining analysis of CD49a+ T_{RM} demonstrated decreased IL17a production relative to CD103+ T_{RM} regardless of treatment, but only increased IFNγ production in mice that received either vaccinia or FluMist® vaccination. While it is possible that IFNγ production is required for eliciting protective effects from vaccinations, the intracellular cytokine staining analyses of T_{RM}s recovered from bleomycin-treated lungs, it also apprears CD49a+CD4+ T_{RM} may contribute to the protective effect of vaccination on fibrosis development in the lung.

### Example 3

Determination that CD49a+ CD4+ T_{RM} Cells are Protective Against Pulmonary Fibrosis

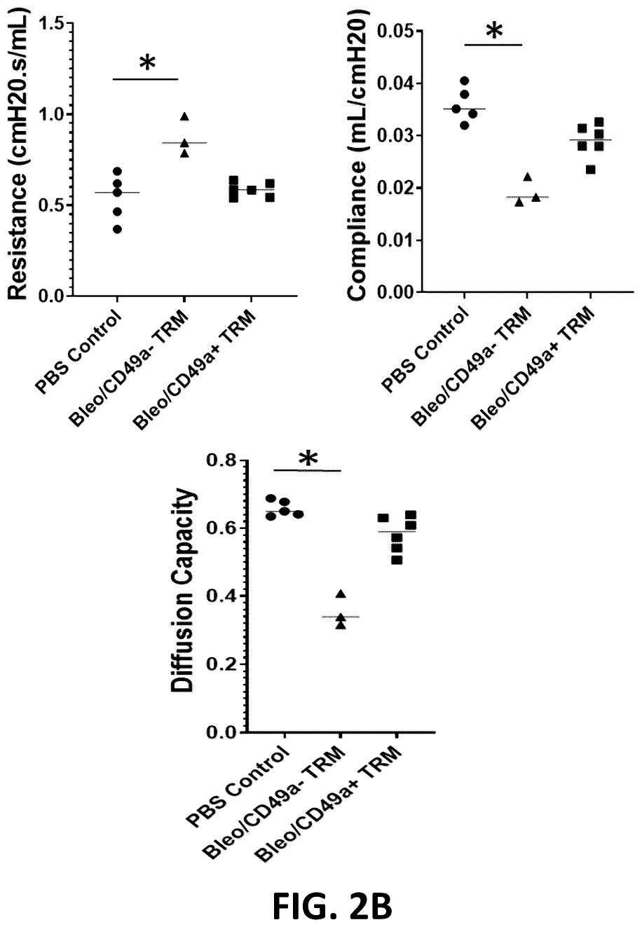

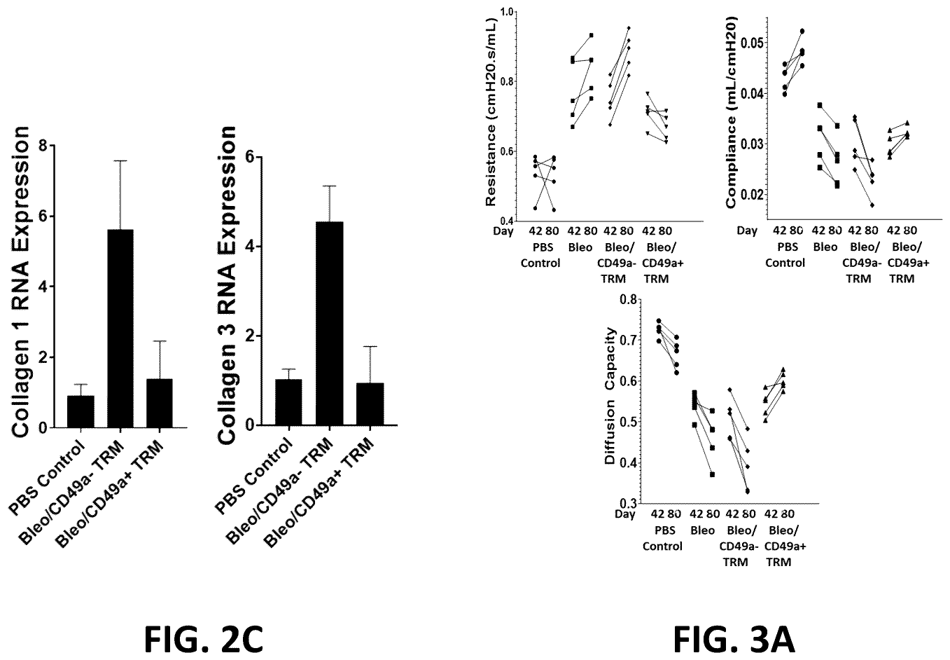

This example covers the protective effects of CD49a+CD4+ T_{RM} against pulmonary fibrosis. The protective effects of multiple T_{RM} phenotypes were characterized following administration to pulmonary fibrotic mice. Pulmonary fibrosis was induced in a first mouse cohort with intraperitoneal bleomycin injections on days 0, 3, 7, 14, 21, and 28. Unlike the intratracheal model of pulmonary fibrosis this model results in long term progressive lung fibrosis which better mimics human disease. T_{RM} were generated through intranasal vaccination with FluMist® of a second mouse cohort, and collected 28 days later with sorting into CD49a+ CD4+ and CD49a− CD4+ subpopulations (FIG. 2A).

42 days after the initial bleomycin treatment, 50,000 sorted CD49a+ or CD49− CD4+ T cells were intratracheally administered to the IP bleomycin treated mouse cohort. After another 30 days (on day 72), the mice were subjected to pulmonary function testing. Mice which received CD49a− CD4+ T_{RM} demonstrated significantly increased tissue resistance and significantly decreased compliance and diffusion capacity of carbon monoxide (CO) relative to PBS treated control mice (FIG. 2B), reflecting limited ability of the T_{RM} to inhibit lung fibrosis. In contrast, mice which received CD49a+ CD4+ T_{RM} demonstrated no notable change in tissue resistance, lung compliance or diffusion capacity relative to PBS control mice, indicating a protective role for these T_{RM} against fibrosis development.

Lungs of the CD49a−CD4+ T_{RM} and CD49a+CD4+ T_{RM} treated mice were also subjected to RNA analysis for collagen expression. Mice which received CD49a− CD4+ T_{RM} exhibited significantly increased levels of collagen 1 and collagen 3 in the lungs. Contrasting this group, mice which received CD49a+ CD4+ T_{RM} did not demonstrate a significant increase in collagen 1 or collagen 3 relative to PBS treated mice (FIG. 2C). These data further support a protective role for CD49a+ CD4+ T_{RM} against the development of pulmonary fibrosis.

### Example 4

### Reversal of Pulmonary Fibrosis in T_{RM} Cell-Treated Mice

To further confirm the protective role of CD49a+ CD4+ T cells in our model of bleomycin and to rule out unforeseen effects on mouse lung health and other sources of variation stemming from bleomycin injections, mice with pulmonary fibrosis and T_{RM}s were monitored over multiple months.

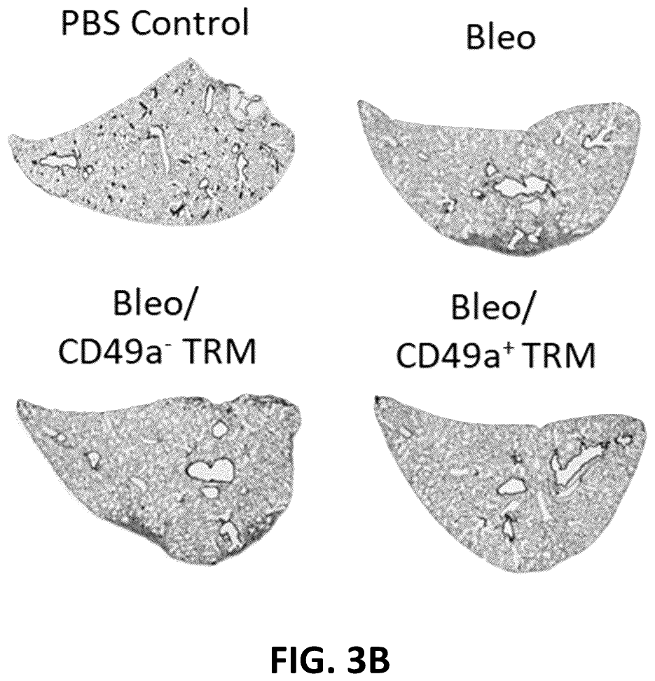

Pulmonary fibrotic mice and T_{RM}s were prepared according to EXAMPLE 2. Baseline pulmonary function testing on day 42 before any adoptive transfer of T_{RM}s. Only mice which demonstrated decreased pulmonary function as determined by increased tissue resistance, decreased lung compliance, and decreased diffusion capacity were selected for CD4+ T cell transfer. On day 49, fibrotic mice received either no CD4+ T cells, 50,000 CD4+ CD49a− T_{RM}, or 50,000 CD49a+ CD4+ T_{RM} intratracheally. Mice were followed for 31 more days and on Day 80, pulmonary function testing was again performed. Mice that received bleomycin without T cell transfer or bleomycin followed by CD49a−CD4+ T_{RM} demonstrated increased resistance and decreased lung compliance and diffusion capacity relative to their function observed at day 42 and to PBS controls, consistent with the continued development of lung fibrosis (FIG. 3A). However, mice which received bleomycin followed by CD49a+ CD4+ T_{RM} demonstrated decreased tissue resistance and increased lung compliance and diffusion capacity at day 80 relative to their function observed at day 42, reflecting improved lung function between days 42 and 80 and indicating reversal of the progressive fibrosis seen in the other experimental conditions.

Histology of lungs isolated at day 100 mirrored the pulmonary function data, with lungs from mice which received no T cells or CD49a− CD4+ T_{RM} demonstrating increased collagen deposition and cellular infiltration, and lungs from mice which received CD49a+ CD4+ T_{RM} resembling lungs from PBS treated mice (FIG. 3B). These data indicate that even after the initiation of lung fibrosis as determined by increased resistance, and decreased compliance, and diffusion capacity observed at day 42, CD49a+ CD4+ T_{RM} are capable of halting and even reversing the development of pulmonary fibrosis.

### Example 5

### Human CD49a+ CD4+ T Cells Suppress Profibrotic Gene Expression

Building off of the findings of EXAMPLES 1-3 that CD49a+ CD4+ T cells can protect against and reverse pulmonary fibrosis, the roles of T_{RM}s were analyzed in models of human idiopathic pulmonary fibrosis (TPF). Coculture experiments were performed with human CD4+ T cells and lung fibroblasts cultured from biopsies of IPF patients. As lung fibroblasts are known to be significant producers of collagen in fibrotic lungs, it was of particular interest whether T_{RM}s would affect similar decreases in collagen 1 and 3 expression as in the mouse models of EXAMPLES 1-3.

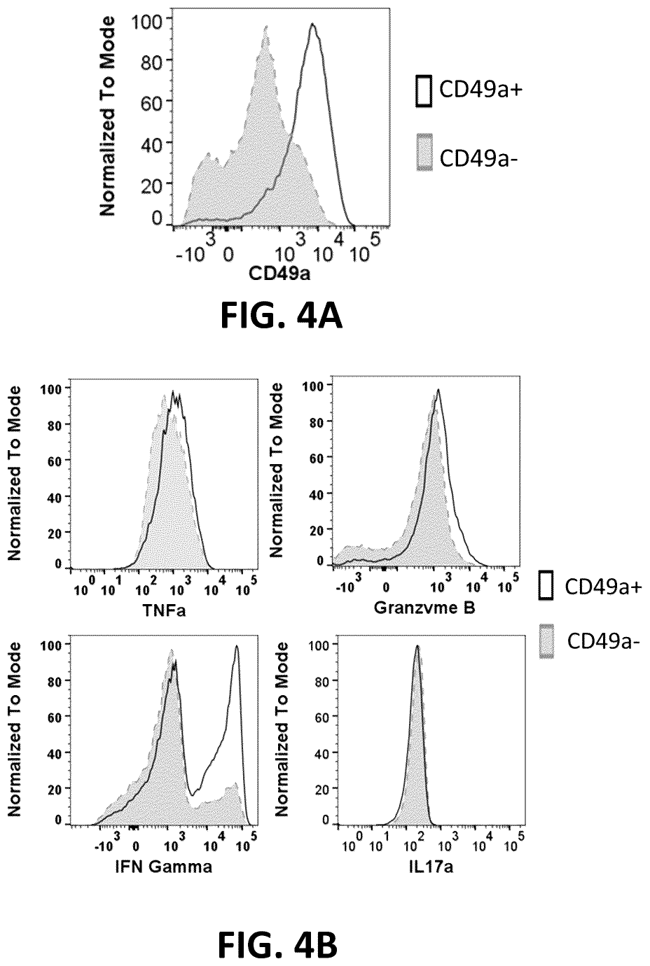

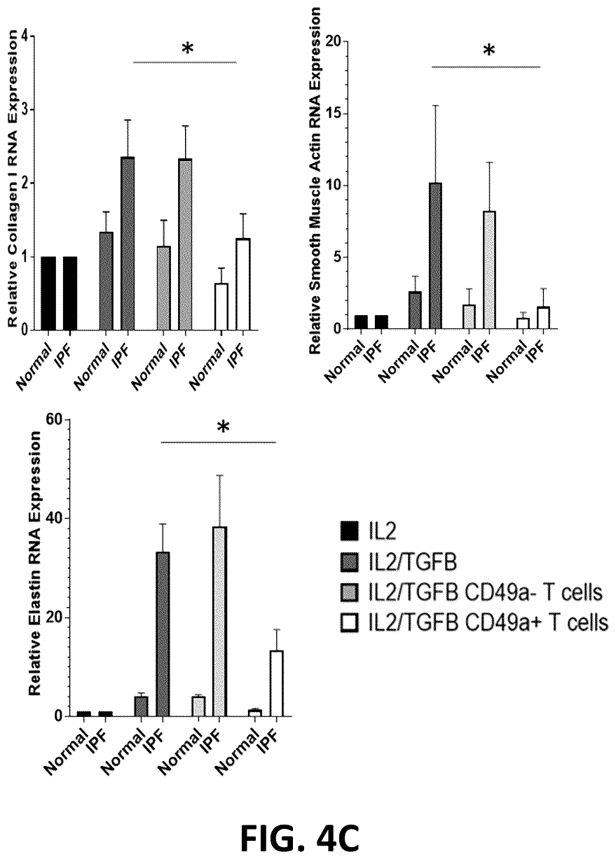

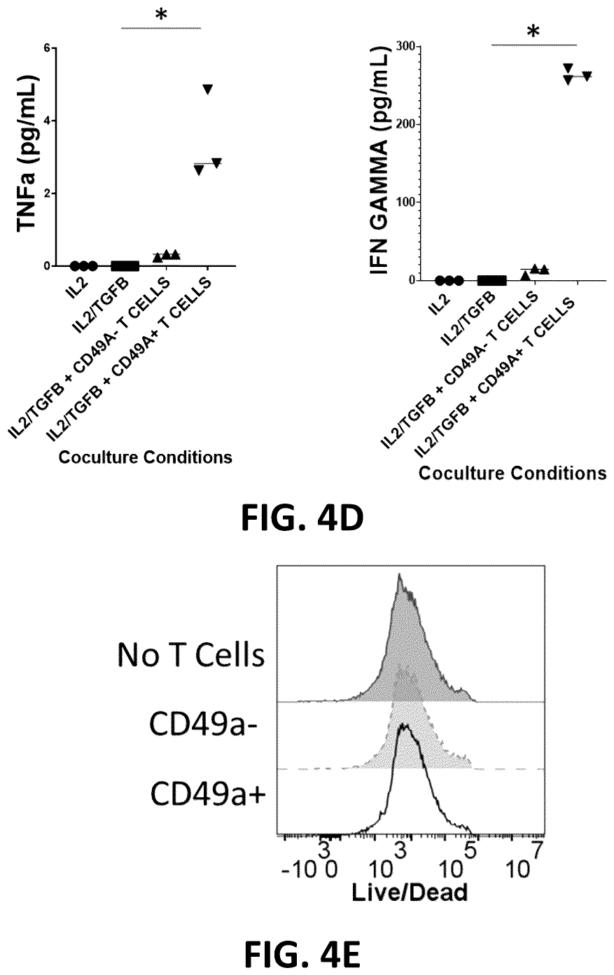

To generate CD49a+ CD4+ T cells, human PBMCs were stimulated with anti-CD3 and anti-CD28 in the presence of interleukin-2 for four weeks. CD4+ T cells were magnetically isolated and then magnetically separated based on CD49a expression. Stimulation of PBMCs resulted in the upregulation of CD49a on approximately 25% of isolated CD4+ T cells, which were effectively isolated by magnetic separation (FIG. 4A).

CD49a−CD4+ and CD49a+CD4+ T cells were stimulated with PMA and Ionomycin and subjected to ICS analysis. Functionally CD49a+ CD4+ T cells produced more of the classical Th1 cytokines TNFα, granzyme b, and IFNγ than CD49a− CD4+ T cells, while neither CD49a− or CD49a+ T cells produced significant amounts of IL17a (FIG. 4B).

To assess the effect of the isolated CD4+ T cells on the IPF fibroblasts, transwell coculture experiments were performed in which CD4+ T cells were added at a 2:1 ratio with either normal lung fibroblasts or lung fibroblasts from IPF patients. IL2 was added to stimulate the CD4+ T cells and cells were incubated overnight before the addition of TGFβ to induce profibrotic gene expression.

Fibroblast RNA was collected after 24 hours of TGFβ stimulation. Addition of TGFβ induced a significant increase in collagen 1, smooth muscle actin, and elastin gene expression, with IPF fibroblasts producing significantly greater amounts of these transcripts than normal fibroblasts (FIG. 4C). Coculture with CD49a− CD4+ T cells had no effect on profibrotic gene expression relative to TGFβ addition alone. However, coculture with CD49a+ CD4+ T cells resulted in a significant reduction in profibrotic gene expression supporting a protective role for these T cells against the onset and progression of fibrosis.

Coculture supernatants were assayed for cytokines. Increased levels of TNFα and IFNγ were observed in wells for which CD49a+ CD4+ T cells were added. While overall TNFα levels were barely detectable, the levels of IFNγ were significantly increased in wells which contained CD49a+ CD4+ T cells, paralleling the T_{RM} ICS analyses (FIG. 4D).

Further analyses were performed on the IPF fibroblasts to determine whether the observed decrease in profibrotic gene expression was due to killing T_{RM}-mediated cell death. IPF fibroblasts were isolated post coculture, stained with a live/dead dye, and identified as alive or dead through flow cytometry. In spite of the fact that increased granzyme b levels were observed following CD49a+ CD4+ T cell stimulation, the flow cytometry measurements determined that coculture with either CD49a− or CD49a+ CD4+ T cells did not result in increased death of IPF fibroblasts (FIG. 4E).

Collectively, the gene expression profiling, ICS, and flow cytometry data support a protective role for CD49a+ CD4+ T cells against fibrotic development and specifically demonstrate the ability of human CD49a+ CD4+ T cells to suppress profibrotic gene expression by IPF fibroblasts.

### Example 6

### CD49a+ T_{RM} Cells Alter the Fibrotic Lung Environment

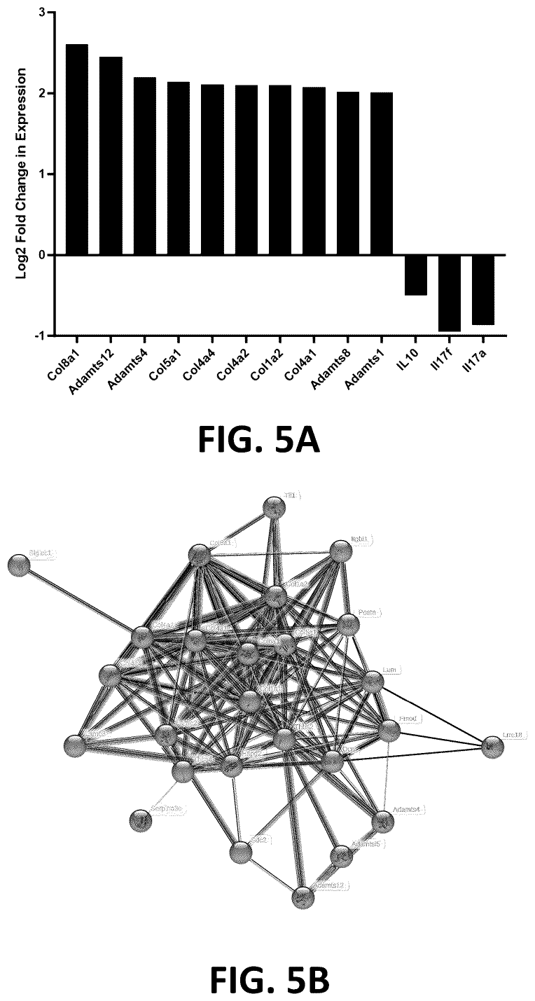

This example concerns potential mechanisms by which T_{RM} cells improve lung function, reverse pulmonary fibrosis, and modify the lung environment. Mouse T_{RM} cells were generated and characterized according to EXAMPLE 2. RNA sequence analysis was then performed on CD49a+CD4+ and CD49a−CD4+ T_{RM} cells. To identify upregulated molecular pathways, the top 200 most significantly upregulated differentially expressed genes by CD49a+ CD4+ T cells were subjected to STRING analysis of known and predicted protein-protein interactions. Clustering of upregulated proteins resulting in a cluster which contained 26 upregulated genes with a small PPI enrichment p-value (<10⁻¹⁶), indicating a high likelihood that the proteins are biologically connected (FIG. 5B), namely Adamts4, Adamts5, Adamts12, Col1a1, Col1a2, Col4a1, Col4a2, Col4a4, Col5a1, Col8a1, Dcn, Fmod, Hspg2, itgbl1, Lama2, Lamc3, Lum, Postn, Sdc2, Serpina3c, Siglec1, Thbs2, Thbs4, Tll1. The identified cluster contained many proteins involved in collagen-activated tyrosine kinase receptor signaling, extracellular matrix organization, platelet-derived growth factor binding, collagen binding, heparin binding, and glycosaminoglycan binding. In addition, the cluster contained several proteases and metallopeptidases. Differential gene analysis comparing lung CD49a+ CD4+ TRM to lung CD49a− CD4+ TRM demonstrated significantly increased expression of genes involved in the extracellular matrix such as collagens 4, 5, and 8, as well as the extracellular modifying genes Adamts1, 4, 8, and 12. In addition, decreased expression of the cytokines IL17a and IL17f was observed (FIG. 5A).

These data suggest that CD49a⁺CD4⁺ T_{RM} cells may act directly on the fibrotic extracellular matrix. Potentially binding to deposited collagen and upregulated platelet derived growth factor receptor. Through this binding, CD49a+ CD4+ T cells may be inducing degradation of collagen and other extracellular matrix proteins present in increased quantities during fibrosis.

### Example 7

### CD49a+ CD4+ T Cell Populations in Human IPF Lungs

In EXAMPLES 1-5, it was determined that human CD49a+ CD4+ T cells are capable of suppressing profibrotic gene expression in vitro and that transfer of CD49a+ CD4+ T cells can reverse established fibrosis in bleomycin treated mice. It was further determined that bleomycin treated mice have increased proportions of CD103+CD4+ T_{RM} cells relative to CD49a+CD4+ T_{RM} cells, whereas control and vaccinated mice have increased ratios of CD49a+ CD4+ to CD103+CD4+ T_{RM} cells (data not shown). These findings indicate a skew of lung T_{RM} towards a pathogenic CD103+ phenotype and away from a protective CD49a+ phenotype following the onset of fibrosis. Based on these findings, it was hypothesized that human IPF would affect a similar skew in CD4+ T cell phenotypes.

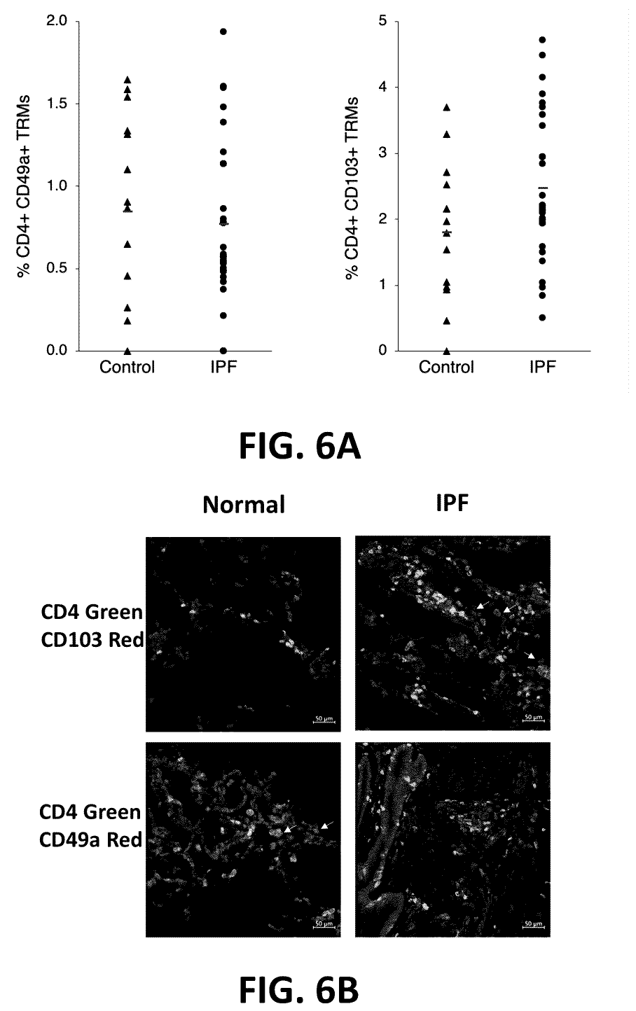

The possible presence of these cells in human lungs was also assessed by analyzing scRNAseq data from fibrotic human lungs obtained from the Gene Expression Omnibus database (accession number GSE135893). Analysis of the data from annotated T cells identified overall low expression levels of CD49a⁺ TRMs. However, there were fewer CD49a⁺ TRMs and more CD103⁺ TRMs in the IPF lungs compared to control lungs (FIG. 6A), supporting the possibilities that these cells are present in human lungs and are decreased in fibrotic lungs.

To further assess the presence of these cells in healthy and diseased lungs, immunofluorescence was performed on lung biopsy samples from age matched normal and IPF patients. IHC of CD4 and CD103 demonstrated few CD103+ CD4+ T cells in normal lungs (FIG. 6B). However, IHC of IPF lungs identified high levels of CD4+ T cells, many of which co-stained with CD103. In contrast, IHC of CD4 and CD49a demonstrated populations of CD49a+ CD4+ T cells in normal lungs, but few CD4+ T cells co-staining with CD49a in the lungs of IPF patients. These data strongly support a skewing of lung T_{RM} away from a protective CD49a+ phenotype and toward a pathogenic CD103+ phenotype in human IPF, consistent with the data obtained from mouse models of bleomycin induced fibrosis. These data also highlight the potential for therapies which induce a protective CD49a+ phenotype in lung T_{RM}.

Although the invention has been described with reference to the above examples, it will be understood that modifications and variations are encompassed within the spirit and scope of the invention. Accordingly, the invention is limited only by the following claims.

What is claimed is:

1. A method of treating fibrosis in a subject in need thereof comprising administering to the subject a composition comprising a T resident memory (T_{RM}) cell, thereby treating the fibrosis.

2. The method of claim 1, wherein the T_{RM} cell is CD4⁺.

3. The method of claim 1, wherein the T_{RM} cell is CD49a⁺.

4. The method of claim 1, wherein the T_{RM} cell does not express CD103.

5. The method of claim 1, wherein the T_{RM} cell is CD69⁺, TIM-3⁺, IFNγ⁺, or a combination thereof.

6. The method of claim 1, wherein the T_{RM} cell is derived from the subject.

7. The method of claim 1, wherein genes in the T_{RM} cell are upregulated following administration of the T_{RM} cell to the subject.

8. The method of claim 7, wherein the genes encode proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins.

9. The method of claim 7, wherein the genes are selected from Adamts4, Adamts5, Adamts12, Col1a1, Col1a2, Col4a1, Col4a2, Col4a4, Col5a1, Col8a1, Dcn, Fmod, Hspg2, itgbl1, Lama2, Lamc3, Lum, Postn, Sdc2, Serpina3c, Siglec1, Thbs2, Thbs4, Tll1, or a combination thereof.

10. The method of claim 1, wherein the fibrosis is in a tissue selected from lung, liver, kidney, heart, muscle, or brain.

11. The method of claim 10, wherein the fibrosis is in the lung.

12. The method of claim 11, wherein the lung fibrosis is selected from chronic hypersensitivity pneumonitis, idiopathic pulmonary fibrosis, silicosis, radiation pneumonitis, collagen vascular disease, or a combination thereof.

13. The method of claim 1, wherein the administering comprises intratracheal administration, intrabronchial administration, intranasal administration, nebulization, powder inhalation, intravenous injection, intrapulmonary injection, intraperitoneal, intrathecal, or pulmonary artery infusion.

14. The method of claim 13, wherein the administering comprises intratracheal administration or intranasal administration.

15. The method of claim 1, wherein the composition comprises between about 5×10³ to 5×10⁶ cells.

16. The method of claim 1, further comprising repeating the administering after about 1 to 400 days.

17. The method of claim 1, further comprising contacting a peripheral blood mononuclear cell (PBMC) with a CD3 antibody, a CD28 antibody, and IL2, thereby generating the T_{RM} cell.

18. The method of claim 17, further comprising isolating the T_{RM} cell with a CD4 antibody, a CD49a antibody, or a combination thereof.

19. A method of increasing a proportion of CD49a⁺CD4⁺ to CD103⁺CD4⁺ cell phenotypes in a tissue of a subject in need thereof comprising administering to the subject, a composition comprising a CD49a⁺CD4⁺ T_{RM} cell to the subject, thereby increasing the proportion of CD49a⁺CD4⁺ to CD103⁺CD4⁺ cell phenotypes in a tissue of the subject.

20. A pharmaceutical composition comprising a CD49a⁺ T resident memory (T_{RM}) cell formulated in a carrier suitable for inhalation or nebulization.

21. The composition of claim 20, wherein the CD49a⁺ T_{RM} cell is a CD4⁺ cell.

22. The composition of claim 20, wherein the CD49a⁺ T_{RM} cell does not express CD103.

23. The composition of claim 20, wherein the CD49a⁺ T_{RM} cell is a CD69⁺ cell, an IFNγ⁺ cell, aTIM-3⁺ cell, or a combination thereof.

24. The composition of claim 20, wherein genes encoding proteins selected from collagen-activated tyrosine kinase receptor signaling proteins, extracellular matrix organization proteins, platelet-derived growth factor binding proteins, collagen binding proteins, heparin binding proteins, and glycosaminoglycan binding proteins are upregulated in the CD49a⁺ T_{RM} cell.

25. The composition of claim 20, wherein genes selected from Adamts4, Adamts5, Adamts12, Col1a1, Col1a2, Col4a1, Col4a2, Col4a4, Col5a1, Col8a1, Dcn, Fmod, Hspg2, itgbl, Lama2, Lamc3, Lum, Postn, Sdc2, Serpina3c, Siglec1, Thbs2, Thbs4, Tll1, or a combination thereof are upregulated in the CD49a⁺ T_{RM} cell.

26. The composition of claim 20, wherein IL17a, PD-1, or a combination thereof is downregulated in the CD49a⁺ T_{RM} cell relative to a CD49a−CD4+ T cell.

27. The composition of claim 20, wherein the pharmaceutical composition comprises between about 5×10³ to 5×10⁶ cells.
